(19) United States
(12) Patent Application Publication
(10) Pub. No.: US 2026/0345701 A1
(43) Pub. Date: Nov. 5, 2026

(54) PROBIOTIC COMPOSITIONS AND THEIR USE TO MODULATE IMMUNE SYSTEMS

(71) Applicant: SYNBALANCE SRL, Origgio (VA) (IT)

(72) Inventors: Rosella MALANCHIN, Origgio (VA) (IT); Cristiana PIANGIOLINO, Origgio (VA) (IT); Silvia CASTEGNARO, Origgio (VA) (IT); Federica CARLOMAGNO, Origgio (VA) (IT); Chiara PESCIAROLI, Origgio (VA) (IT)

(21) Appl. No.: 19/721,896

(22) Filed: Jun. 26, 2026

Related U.S. Application Data
(62) Division of application No. 18/017,037, filed on Jul. 29, 2021.

(30) Foreign Application Priority Data
Jul. 31, 2020 (IT) 102020000018895

(51) Int. Cl. A61K 35/747; A61K 9/00; A61K 9/48; A61K 31/355; A61K 31/4415; A61K 31/519; A61K 31/593; A61K 31/714; A61K 35/745; A61K 47/26; A61K 47/36; A61K 47/44; A61P 31/00; A61P 37/04

(52) U.S. Cl. CPC A61K 35/747; A61K 9/0046; A61K 9/0053; A61K 9/4866; A61K 31/355; A61K 31/4415; A61K 31/519; A61K 31/593; A61K 31/714; A61K 35/745; A61K 47/26; A61K 47/36; A61K 47/44; A61P 31/00; A61P 37/04

(57) ABSTRACT

Disclosed are probiotic compositions including: one or more strains belonging to the genus Lactobacillus selected from the species: Lactobacillus plantarum and Lactobacillus acidophilus; and one or more strains belonging to the genus Bifidobacterium selected from the species: Bifidobacterium lactis, Bifidobacterium infantis and Bifidobacterium longum. The compositions include: at least one strain of Lactobacillus plantarum and at least one strain of Bifidobacterium lactis; and at least two strains belonging to the genus Lactobacillus or to the genus Bifidobacterium as defined above. Also disclosed are methods to modulate immune systems using such compositions.

### CROSS-REFERENCE TO RELATED APPLICATIONS

This application is a Divisional of application Ser. No. 18/017,037, filed on Jan. 19, 2023, which is the National Phase under 35 U.S.C. § 371 of International Application No. PCT/IB2021/056908, filed on Jul. 29, 2021, which claims the benefit under 35 U.S.C. § 119(a) to patent application No. 102020000018895, filed in Italy on Jul. 31, 2020, all of which are hereby expressly incorporated by reference into the present application.

### BACKGROUND OF THE INVENTION

### Field of the Invention

The present invention relates to probiotic compositions comprising one or more strains belonging to the genus Lactobacillus and one or more strains belonging to the genus Bifidobacterium, wherein said compositions comprise at least one strain of Lactobacillus plantarum and at least one strain of Bifidobacterium lactis, provided that at least two strains belonging to the genus Lactobacillus or to the genus Bifidobacterium are present in the composition, useful to modulate immune systems.

### Description of the Related Art

The immune system is the most important barrier dedicated to the defense of the organism from pathogens to the repair of tissue damages and guarantees continuous surveillance. The immune system begins to develop from birth and fortify itself in childhood: about 80% of the plasma cells capable of producing IgA are associated with the intestinal mucosa, and 70% of the immune system cells are present in the lymphoid tissue associated with the intestine or GALT.

Seasonal flu is mainly contracted by air, through saliva or respiratory secretions released into the air by carriers of infection. It is also possible to contract flu through surfaces or objects contaminated and bringing the hands to the mouth, nose or eyes. The main responsible for seasonal flu are two viruses: type A and B.

Although vaccines are available, seasonal infections cause between 3 and 5 million potentially fatal cases and about 250,000 deaths worldwide. Specific pharmacological treatments for flu are not available with the only exception of pain relievers and antipyretics which do not act on the virus but on the associated symptoms.

Chronic fatigue syndrome, or myalgic encephalomyelitis, is a chronic, multifactorial and debilitating disease mainly characterised by immunological changes. The onset of this disease is often sudden and characterised by profound fatigue accompanied by cognitive dysfunction, sleep disturbances, pain and other symptoms that are worsened by any kind of exertion. The age group most affected is between 20 and 40 years and it is more frequent in women with a 4:1 ratio. The acute manifestation of fatigue is based on a physiological mechanism by which a healthy individual protects himself from different forms of stress; it is generally linked to a single cause and resolves itself with rest and a change in lifestyle. Chronic manifestation, on the other hand, is often associated with previous serious illnesses such as multiple sclerosis, rheumatoid arthritis, etc.

There is currently no specific therapy, but various pharmacological treatments are indicated, such as the administration of immunomodulators, corticosteroids, antivirals or antidepressants combined with a healthy lifestyle.

The high intensity of physical activity plays both a positive and a negative role on the immune system. Compared to a sedentary lifestyle, moderate physical activity brings various benefits to the body by giving greater protection against different types of diseases; on the contrary, extreme and prolonged physical activity causes a transient and generalized depression of the immune function, compromising the body's defenses against potential infections.

Upper airways are the most affected areas: a possible cause is related to the reduction of IgA levels (the main immunoglobulin responsible for protecting the mucous membranes) as well as Natural Killer cells, an important component of innate immunity, which play a key role in viral infections. There are no drug therapies, the only recommended approach is support through a healthy and controlled diet and supplementation.

Some studies have highlighted the effectiveness of probiotics in preventing and supporting the immune system; although the opinion is conflicting, the scientific evidence demonstrating their effectiveness is not sufficiently consolidated.

In the light of the above, there is still a need to identify effective and safe alternative solutions in the prevention and/or treatment of diseases or conditions resulting from an alteration of the immune system.

### SUMMARY OF THE INVENTION

The present invention relates to probiotic compositions comprising:
one or more strains belonging to the genus Lactobacillus selected from the species: Lactobacillus plantarum and Lactobacillus acidophilus;
one or more strains belonging to the genus Bifidobacterium selected from the species: Bifidobacterium lactis, Bifidobacterium infantis and Bifidobacterium longum;
   wherein said compositions comprise:
at least one strain of Lactobacillus plantarum and at least one strain of Bifidobacterium lactis, and
at least two strains belonging to the genus Lactobacillus or to the genus Bifidobacterium as defined above.

Furthermore, the invention relates to the use of said compositions to modulate immune systems, particularly as immunostimulants.

### DESCRIPTION OF THE PREFERRED EMBODIMENTS

The present invention relates to compositions comprising a probiotic combination comprising:
one or more strains belonging to the genus Lactobacillus selected from the species: Lactobacillus plantarum (also known as: Lactiplantibacillus plantarum) and Lactobacillus acidophilus;
one or more strains belonging to the genus Bifidobacterium selected from the species: Bifidobacterium lactis, Bifidobacterium infantis and Bifidobacterium longum;
   wherein said combination comprises:
at least one strain of Lactobacillus plantarum and at least one strain of Bifidobacterium lactis, and
at least two strains belonging to the genus Lactobacillus or to the genus Bifidobacterium as defined above.

It has surprisingly been found that such compositions are effective in modulating the immune defenses, particularly as immunostimulants, in particular in adults and the elderly.

According to the present invention, the immune defenses are positively modulated, meaning that the activity of the immune system is regulated, so that the human body is able to provide an effective immune response against foreign agents.

According to a further preferred embodiment of the invention, the strains belonging to the genus Lactobacillus of the species Lactobacillus plantarum and Lactobacillus acidophilus are selected from Lactobacillus plantarum PBS067 and Lactobacillus acidophilus PBS066 and the strains belonging the genus Bifidobacterium belonging to the species: Bifidobacterium lactis, Bifidobacterium infantis, Bifidobacterium longum are selected from Bifidobacterium lactis BL050, Bifidobacterium infantis BI221 and Bifidobacterium longum BLG240.

Lactobacillus plantarum strain (L. plantarum o LP) called “PBS067” was deposited at the Leibniz-Institut DSMZ-Deutsche Sammlung von Mikroorganismen und Zellkulturen GmbH, under the Budapest Treaty, on 17 Jun. 2011 under Accession Number “DSM 24937”.

Lactobacillus acidophilus strain (L. acidophilus o LA) called “PBS066” was deposited at the Leibniz-Institut DSMZ-Deutsche Sammlung von Mikroorganismen und Zellkulturen GmbH, under the Budapest Treaty, on 17 Jun. 2011 under Accession Number “DSM 24936”. Bifidobacterium lactis strain (B. lactis o BL) called “BL050” was deposited at the Leibniz-Institut DSMZ-Deutsche Sammlung von Mikroorganismen und Zellkulturen GmbH, under the Budapest Treaty, on 17 Jan. 2012 under Accession Number “DSM 25566”.

Bifidobacterium infantis strain (B. infantis o BI) called “BI221” was deposited at the BCCM (Belgian Coordinated Collections of Micro-organisms)—LMG (Laboratorium voor Microbiologie—Bacteriënverzamelig under the Budapest Treaty, on 24 May 2016 under Accession Number “LMG P-29639”.

Bifidobacterium longum strain (B. longum o BLG) called “BLG240 was deposited at the BCCM (Belgian Coordinated Collections of Micro-organisms)—LMG (Laboratorium in voor Microbiologie—Bacteriënverzamelig under the Budapest Treaty, on Aug. 4, 2016 under Accession Number “LMG P-29511”.

According to a further aspect, the compositions comprise as active ingredients, a probiotic combination comprising:
one or more strains belonging to the genus Lactobacillus selected from the species: Lactobacillus plantarum and Lactobacillus acidophilus;
one or more strains belonging to the genus Bifidobacterium selected from the species: Bifidobacterium lactis, Bifidobacterium infantis and Bifidobacterium longum;
   wherein said combination comprises:
at least one strain of Lactobacillus plantarum and at least one strain of Bifidobacterium lactis, and
at least two strains belonging to the genus Lactobacillus or to the genus Bifidobacterium as defined above, with at least one physiological acceptable excipient and/or carrier.

The compositions may be formulated by conventional methods. Preferred forms of administration are solid formulations, such as hard capsules, sachets, sticks, oro-soluble sticks, tablets, granules, or liquid formulations, such as vials with dosing cap mono- or multi-dose, multi-dose dispersions in oily phase, drops, syrups, multi-phase emulsions, etc.

Lactobacillus plantarum (LP), preferably the strain Lactobacillus plantarum PBS067, may be present in the composition as a percentage by weight on the total weight of the probiotic combination from 0.5% to 30%, preferably from 5% a 25%, even more preferably it is equal to 8% and 20%. Lactobacillus plantarum (LP), preferably the strain Lactobacillus plantarum PBS067, may be present in each individual unit dose in amounts ranging from 0.5 to 2.5 billion CFU, preferably in amounts ranging from 1 to 2 billion CFU, more preferably in amount of 1 billion CFU.

Lactobacillus acidophilus (LA), preferably the strain Lactobacillus acidophilus PBS066, may be present in the composition as a percentage by weight on the total weight of the probiotic combination from 5% to 55%, preferably from 15% a 50%, even more preferably it is equal to 45% and 21%. Lactobacillus acidophilus (LA), preferably the strain Lactobacillus acidophilus PBS066, may be present in each individual unit dose in amounts ranging from 0.5 to 2.5 billion CFU, preferably in amounts ranging from 1 to 2 billion CFU, more preferably present in amount of 1 billion CFU.

Bifidobacterium lactis (BL), preferably the strain Bifidobacterium lactis BL050, may be present in the composition as a percentage by weight on the total weight of the probiotic combination from 5% a 45%, preferably from 10% a 40%, even more preferably it is equal to 35% and 13%. Bifidobacterium lactis (BL), preferably the strain Bifidobacterium lactis BL050, may be present in each individual unit dose in amounts ranging from 0.5 to 2.5 billion CFU, preferably in amounts ranging from 1 to 2 billion CFU, more preferably present in amount of 1 billion CFU.

Bifidobacterium infantis (BI), preferably the strain Bifidobacterium infantis BI221 may be present in the composition as a percentage by weight on the total weight of the probiotic combination from 25% a 50%, preferably from 30% a 45%, even more preferably it is equal to 37%. Bifidobacterium infantis (BI), preferably the strain Bifidobacterium infantis BI221, may be present in each individual unit dose in amounts ranging from 0.5 to 2.5 billion CFU, preferably in amounts ranging from 1 to 2 billion CFU, more preferably present in amounts of 1 billion CFU.

Bifidobacterium longum (BLG), preferably the strain Bifidobacterium longum BLG240, may be present in the composition as a percentage by weight on the total weight of the probiotic combination from 30% a 50%, preferably from 35% a 45%, even more preferably it is equal to 37 and 42%. Bifidobacterium longum (BLG), preferably the strain Bifidobacterium longum BLG240, may be present in each individual unit dose in amounts ranging from 0.5 to 2.5 billion CFU, preferably in amounts ranging from 1 to 2 billion CFU, more preferably present in amount of 1 billion CFU.

Species present in the probiotic combination, as reported in Example 1, may be present in a weight ratio of 1:1.75:5:5 or 1:1:1:1 ratio when expressed in CFU.

Species present in the probiotic combination, as reported in Example 2, may be present in a 1:2.5:1.75 weight ratio or 1:1:1 ratio when expressed in CFU.

Probiotic compositions of the invention can be administered orally or topically (such as by nasal or auricular route).

A further object of the present invention is the use of probiotic compositions, able to modulate the immune system (in particular to stimulate and strengthen the immune system), in the prevention and/or treatment of viral infections, such as seasonal infections, such as flu, cold and upper respiratory tract infections.

In addition, a further object of the invention is the use of probiotic compositions, which are capable of modulating the immune system, in particular as immunostimulants in athletes, particularly in the prevention and/or treatment of infections in the athlete, particularly if immunodepressed, for example, as a result of intense physical exercise. Intense exercise transiently alters (lowers) certain components of the immune system, leading to depression of the immune system.

According to a preferred aspect, the compositions comprise:
L. plantarum PBS067 and L. acidophilus PBS066, and
B. lactis BL050;
preferably these compositions are used in the prevention and/or treatment of seasonal infections, such as flu, cold, and upper respiratory tract infections in children, adults and professional athletes. The compositions are preferably administered in a dosage of 3 billion CFU/day corresponding to a dosage of 1 billion CFU/day for each strain.

According to a further preferred aspect, the compositions comprise:
L. plantarum PBS067, and
B. lactis BL050, B. infantis BI221 and B. longum BLG240;
preferably these compositions are used to modulate the immune system, particularly in the elderly, and preferably in the prevention and/or treatment of seasonal infections, such as flu and cold, or, as a support, in states of immunosuppression or chronic fatigue. The compositions are preferably administered in a dosage of 4 billion CFU/day corresponding to a dosage of 1 billion CFU/day for each strain.

The examples given below further illustrate the invention.

### EXAMPLES

### Formulation Examples

### Example 1

A composition in the form of a powder stick containing:

| INGREDIENTS | % p/p | BILLION/DOSE |
|---|---|---|
| L. plantarum PBS067 | 0.933 | 1 |
| B. lactis BL050 | 1.555 | 1 |
| B. infantis BI221 | 4.667 | 1 |
| B. longum BLG240 | 4.667 | 1 |
| FOS 93% | 3.600 | — |
| Inulin 90% | 3.703 | — |
| Folic Acid | 0.003 | — |
| Vitamin B12 | 0.035 | — |
| Vitamin B6 | 0.022 | — |
| Sorbitol | 9.000 | — |
| Sucralose | 0.080 | — |
| Cream flavour | 1.300 | — |
| Silicon dioxide | 1.000 | — |
| Maltodextrin | 69.435 | — |

### Example 2

A composition in the form of a powder stick containing:

| INGREDIENTS | % p/p | BILLION/DOSE |
|---|---|---|
| L. plantarum PBS067 | 0.933 | 1 |
| L. acidophilus PBS066 | 2.333 | 1 |
| B. lactis BL050 | 1.555 | 1 |
| FOS 93% | 3.600 | — |
| Inulin 90% | 3.703 | — |
| Folic Acid | 0.003 | — |
| Vitamin B12 | 0.035 | — |
| Vitamin B6 | 0.022 | — |
| Sorbitol | 9.000 | — |
| Sucralose | 0.080 | — |
| Cream flavour | 1.300 | — |
| Silicon dioxide | 1.000 | — |
| Maltodextrin | 76.436 | — |

### Example 3

A composition in the form of oily suspension containing:

| | | |
|---|---|---|
| INGREDIENTS | % p/p | BILLION/DOSE |

Solid phase-cap

| | | |
|---|---|---|
| L. plantarum PBS067 | 6.09 | 1 |
| B. lactis BL050 | 10.14 | 1 |
| B. infantis BI221 | 30.43 | 1 |
| B. longum BLG240 | 30.43 | — |
| Vitamin B3 | 1.48 | — |
| Vitamin B5 | 0.57 | — |
| Vitamin B9 | 0.02 | — |
| Maltodextrin | 20.84 | — |

Liquid phase

| | | |
|---|---|---|
| Sunflower oil | 60.0000 | — |
| Medium-chain triglycerides (MCT) | 38.9990 | — |
| Silicon dioxide | 1.0000 | — |
| Vitamin D3 | 0.0010 | — |

### Example 4

A composition in the form of powder in capsules containing:

| INGREDIENTS | % p/p | BILLION CFU/DOSE |
|---|---|---|
| L. plantarum PBS067 | 3.46 | 1 |
| L. acidophilus PBS066 | 8.64 | 1 |
| B. lactis BL050 | 5.76 | 1 |
| Vitamin B6 | 0.08 | — |
| Vitamin B9 | 0.01 | — |
| Vitamin B12 | 0.14 | — |
| Vegetable magnesium stearate | 1 | — |
| Corn starch | 80.91 | — |

### Example 5

A composition in the form of ear drops containing:

| INGREDIENTS | % p/p | BILLION CFU/DOSE |
|---|---|---|
| L. plantarum PBS067 | 0.471 | 1 |
| L. acidophilusPBS066 | 1.178 | 1 |
| B. lactis BL050 | 0.786 | 1 |
| Sunflower oil | 65.043 | — |
| Medium-chain | 28.908 | — |
| triglycerides (MCT) | | |
| Vitamin E | 3.614 | — |

### EXPERIMENTAL EXAMPLES

The following experimental examples are related to the evaluation of the effectiveness of the compositions of the invention.

### Example 6—Modulation of the Immune System Against Seasonal Infections

### Materials and Methods

A randomized, three arm double-blind, placebo-controlled study was carried out on 75 subjects. Subjects have been randomized in order to be homogeneously divided into three groups to receive the probiotic composition reported in Example 1 or the probiotic composition reported in Example 2 or the placebo formulation (Table 1) once daily for a period of 28 days (T28). Visits were planned as follows: enrollment (T0), end of the treatment (T28) and end of the follow-up period (T56).

The tested formulations were administered to the different groups in forms of sticks containing L. plantarum PBS067, B. lactis BL050, B. infantis BI221, B. longum BLG240 (Example 1) or L. plantarum PBS067, L. acidophilus PBS066 and B. lactis BL050 (Example 2) or placebo (Table 1).

TABLE 1

| INGREDIENTS | % p/p |
|---|---|
| FOS 93% | 3.600 |
| Inulin 90% | 3.703 |
| Folic Acid | 0.003 |
| Vitamin B12 | 0.035 |
| Vitamin B6 | 0.022 |
| Sorbitol | 9.000 |
| Sucralose | 0.080 |
| Cream Flavour | 1.300 |
| Silicon dioxide | 1.000 |
| Maltodextrin | 81.257 |

During the enrollment visit (T0), the volunteers received the study protocol, while during all the scheduled visits (T0, T28, and T56) subjects completed a questionnaire on seasonal symptoms. At the beginning, at the end of the treatment and at the end of follow-up period (T56) salivary (Salivary IgA, salivary TAC) and fecal (β-defensin2) samples were taken for the detection of immunological markers.

### Subjects of the Study

75 subjects of Caucasian origin, of both sexes, with an average age of 69.5±4.9 were enrolled. They were selected according to specific inclusion and exclusion criteria.

Subjects were randomized according to the following 1:1:1 ratio (A, B, C). Both study conductors and study volunteers were unaware of the assigned product. During the treatment period and in the following 28 days after taking the product, there were no side effects or abandonment by the volunteers.

### Assessment of Clinical Parameters

Subjects were evaluated three times during the study: at the beginning (T0), end of the treatment (T28) and at the end of the follow-up (T56). Primary efficacy endpoint was to observe an overall improvement in the selected parameters during the treatment period; as a secondary efficacy endpoint, it was assessed whether these positive effects were also maintained during the period of 28 days after the last formulation intakes (T28-T56).

### Symptoms Questionnaire

During the study, volunteers were asked to fill in a questionnaire regarding their bowel habits and general well-being.

For bowel habits, volunteers could give a score between 0 and −3 representing a significant decrease in the frequency of bowel movements; 0 for normal frequency, and +3 for an increase in frequency.

The score associated with general well-being ranged on a scale from 1 to −2 for poor wellbeing, 0 no change and +3 for a substantial improvement in general well-being. In addition, volunteers were asked to report their symptoms related to the most common ailments on a daily basis in a questionnaire called “Common Infection Disease (CID)” according to FDA guidelines. Adherence to the study by the volunteers was also checked during each scheduled visit.

### Assessment of Fecal β-Defensin2

Fecal β-defensin2 (HBD-2) was measured by enzyme-linked immunosorbent assay according to manufacturer's instructions (Immuno-diagnostic, Bensheim, Germany), the detection limit was 0.077 ng/mL.

### Assessment of Salivary IgA

Saliva samples were centrifuged (at 3000 rpm for 15 min) to collect the supernatant, immediately frozen at −20 °C. Salivary IgA concentration was measured by a commercially available enzyme-linked immunosorbent assay (ELISA) kit (Dia Metra, Milano, Italy). Experiments were carried out according to the manufacturer's protocol, and samples were diluted when necessary.

### Assessment of Total Antioxidant Capacity (TAC) in Salivary Samples Through FRAP

For the bioassay, 3T3 or HT-29 cells were plated in 12 plates at a concentration of 10⁵ cells/plate and incubated with saliva samples, centrifuged in 1 mL DMEM for 24 hours at 37 °C with 5% CO₂.

The enhancement of the antioxidant capacity of the cells in the presence of the salivary samples was evaluated by Ferric Reducing Antioxidant Parameter (FRAP) assay as disclosed by Benzie and Strain. Ferric to ferrous ion reduction at pH 3.6 causes a colored ferrous-2,4,6-tripyridyl-s-triazine (TPTZ) complex. The absorbance at 595 nm of each sample was recorded after 30 min of incubation at room temperature. The absorbance values were compared to a Fe(II) standard curve. Final results are expressed as Fe(II) μM.

### Statistical Methods

The number of days subjects were affected by CID symptoms are represented as average number of days±SEM.

Data of salivary and fecal immunological markers were collected for each volunteer and expressed as an average of the values. As the data were not distributed according to a normal Mann-Whitney-Wilcoxon test, statistically significant differences were observed considering a p<0.05 (*) and p<0.01 (**).

### Results

### Impact on Common Infectious Disease Incidence

During the entire study period, the health status of volunteers was monitored through the daily compilation of Common Infectious Disease (CID). The effectiveness of the two probiotic compositions reported in Examples 1 and 2 on CID was evaluated as the number of subjects who had at least one episode during the entire study period (T0-T56). Results showed that during the entire treatment in the groups that took the probiotic compositions reported in Examples 1 and 2, the percentage of subjects with at least one episode of CID was respectively of 40% in the group treated with the probiotic composition of Examples 1 and 42% in the group treated with the probiotic composition of Examples 1 compared to the 76% of subjects in the placebo group.

The group that took the probiotic composition reported in Example 1 reported an average of 10±2 days (6±1 during the intake of the product and 4±1 in the 28 days following the end of the intake of the product), statistically significant (p<0.01 throughout the study and p<0.05 during the follow-up period) and below the average of days obtained for the placebo group (12±2).

Instead, the group that took the probiotic composition reported in Example 2 showed an average of 5±2 days (3±1 during the intake of the product and 2±1 in the 28 days following the end of the intake of the product), statistically significant and lower than the placebo group (p<0.01). (Table 2)

TABLE 2

| | Total | T0-T28 | T28-T56 |
|---|---|---|---|
| Example 1 | 10 ± 2 | 6 ± 2 | 4 ± 1.0 |
| Example 2 | 5 ± 2 | 3 ± 1 | 2 ± 1 |
| Placebo | 12 ± 2 | 4 ± 1 | 5 ± 1 |

The average number of days of CID in the 28 days following the end of intake of the product, of the group that took the probiotic composition reported in Example 2, is not only lower than the placebo value but also statistically significant (p<0.05) suggesting a long-term beneficial effect even after the end of product intake.

### Analysis of Immunological Markers

Through the selected immunological markers, it was possible to analyse the status of the immune system of volunteers both from a gastrointestinal point of view (Beta-Defensin 2) and respiratory (IgA and TAC).

Analysis of the results obtained for the group taking the probiotic composition reported in Example 1 shows that Beta-Defensin2 values increased significantly (+26%) during the treatment period (T28) and were maintained during the 28 days after the end of the product intake.

The results obtained from the group taking the probiotic composition reported in Example 2 showed a significantly (p<0.05) increase of approximately 22% from the start of treatment (T0-T56). Post-treatment levels were also shown to be statistically significantly higher than placebo (p<0.05). During the entire treatment, the placebo group showed only a slight increase in Beta-Defensin2 values (Table 3).

TABLE 3

| | T0-T28 | T0-T56 |
|---|---|---|
| Example 1 | +26% | +27% |
| Example 2 | +10% | +22% |
| Placebo | +4% | +15% |

In conclusion, the intake of a probiotic composition (Example 1 or 2) led to an effective improvement in the intestinal immune system through positive modulation of the analysed markers.

The immune system is often associated with respiratory tract infections frequently characterized by a reduction of IgA and a subsequent increase of reactive oxygen species that contribute to increase this state of immuno-senescence.

During the study period, a slight improvement in salivary IgA was observed in the group that took the probiotic composition reported in Example 1 during treatment. Instead, the values of the group that took the probiotic composition reported in Example 2 were slightly better compared to both the composition of Example 1 and placebo groups over the whole treatment. In addition, the placebo group was the only one to perceive IgA reduction throughout the study period as observed in Table 4.

TABLE 4

| | T0 | T28 | T56 |
|---|---|---|---|
| Example 1 | 7.32 ± 7.39 | 7.41 ± 5.76 | 5.39 ± 4.51 |
| Example 2 | 8.42 ± 1.45 | 8.59 ± 1.33 | 9.13 ± 1.55 |
| Placebo | 9.29 ± 1.50 | 8.88 ± 1.52 | 8.99 ± 1.50 |

The group that took the probiotic composition reported in Example 1 showed a significant increase in antioxidant capacity during the treatment period (+15% T0-T28 p<0.01), which continued to grow even in the 28 days following the end of treatment.

The evaluation of the antioxidant capacity of the cell, measured by FRAP method, showed variations for the group taking the probiotic composition reported in Example 1 with a significant increase in Total Antioxidant Capacity levels (p<0.05) over the treatment period (T0-T28). In contrast, the placebo group showed a stable trend throughout the study period (Table 5).

TABLE 5

| | T0 | T28 | T56 |
|---|---|---|---|
| Example 1 | 509.20 ± 47.48 | 585.30 ± 55.05 | 598.60 ± 53.68 |
| Example 2 | 562.66 ± 45.34 | 582.57 ± 41.52 | 606.01 ± 51.20 |
| Placebo | 527.50 ± 40.01 | 540.69 ± 42.38 | 511.03 ± 38.58 |

In conclusion, the intake of the probiotic compositions reported in Example 1 or 2 has been shown to positively modulate the immune response related to the respiratory tract, but to the immune system in general.

### Example 7—Gut Microbiota Analysis

### Gut Microbiota Analysis Related to the Probiotic Composition Reported in the Example 1

Among the taxa enhanced by the intake of the probiotic composition reported in Example 1, after 28 days it was evident the presence of genera that are reported to be beneficial for the host. The Lachnospiraceae family of the Firmicutes phylum was increased during the treatment in terms of biodiversity. This genus is very well known to be Short Chain Fatty Acids (SCFAs) producer, especially butyrate, being able to boost anti-inflammatory capacity of host, by suppressing activation of pro-inflammatory pathways. Likewise, the genus Bifidobacterium was enhanced by the treatment. It has been observed also a positive variation in terms of biodiversity of Bacteroides genus which is known for the beneficial conversion of succinate to propionate and for the production of sphingolipids important for keeping the homeostasis and modulating inflammation. Dialister genus was particularly enhanced by probiotics intake over the treatment. Positive variations due to probiotics administration were observed, and still evident after the wash-out period, for genera such as Christensenellaceae, Lachnoclostridium. and Butyricicoccus. A member of the genus Butyricicoccus, as the species B. pullicaecorum can also colonize the mucus layer of the human colon, enhancing the anti-inflammatory effect, furthermore it is also capable to produce butyric acid.

### Gut Microbiota Analysis Related to the Probiotic Composition Reported in the Example 2

Bacterial diversity of the two groups (alpha-diversity, active group vs. placebo) was used to estimate the changes of biodiversity within each treatment-group at the three time-points. Unlike placebo, probiotic composition reported in the Example 2 (active group) showed significant differences between the initial and the final time-points T0-T56 (p-value<0.05). These results indicated that the probiotic group led to a higher biodiversity after T28 that is maintained until the end of the wash-out period (T56).

In order to evaluate the variation of microbiota biodiversity between groups, the microbiota maturity was investigated indicating an increase of the community richness in active group compared to placebo after the intake period (T28) which is maintained until the end of the wash-out period.

The analysis revealed that the active treatment positively influenced the presence of Faecalibacterium to a highly stable frequency for all the duration of the study, while no significant variation was observed for the placebo group. Regarding the effects of the probiotics intake on the most relevant taxa, bacterial genera Akkermansia, Bifidobacterium, Blautia, Faecalibacterium, Prevotella, Roseburia, and Ruminococcus were considered for their highest rate of variation and for their relevance in inducing gut metabolism changes. These genera demonstrated to have a higher positive cumulative variation after probiotic intake that increased during all the study period, while the placebo treatment induced a decreasing trend. To study the effect of probiotic intake on several species, the relative abundances were investigated showing an increase in the number of Bifidobacterium spp. during the treatment (T28). Furthermore, the quantity of bacteria belonging to the same species of the probiotics administered (L. plantarum, L. acidophilus and B. lactis) was assessed showing an increasing trend in the amount of these bacteria in synbiotic group.

In particular, at time T28, a significantly higher amount of L. plantarum and B. lactis compared to time T0 and to placebo was evidenced (p-value<0.01 and p-value<0.05 respectively). The amount of L. acidophilus was stable and statistically higher with compared to placebo (p-value<0.01).

1. A method for preventing and/or treating a viral infection, comprising administering an effective dose of a composition to a patient in need thereof, wherein the composition comprises a probiotic combination comprising:
Lactobacillus plantarum PBS067, Lactobacillus acidophilus PBS066, and Bifidobacterium lactis BL050; and
at least one physiologically acceptable excipient and/or carrier,
wherein:
the Lactobacillus plantarum PBS067 is present in the composition in a percentage by weight on the total weight of the probiotic combination from 0.5% to 30%;
the Lactobacillus acidophilus PBS066 is present in the composition in a percentage by weight on the total weight of the probiotic combination from 5% to 55%; and
the Bifidobacterium lactis BL050 is present in the composition in a percentage by weight on the total weight of the probiotic combination from 5% to 45%.

2. The method of claim 1, wherein the patient is selected from the group consisting of adults, the elderly, and athletes.

3. The method of claim 1, wherein the composition is administered orally or topically.

4. A method for preventing and/or treating a viral infection in an immunodepressed patient, comprising administering an effective dose of a composition to the immunodepressed patient, wherein the composition comprises a probiotic combination comprising:
Lactobacillus plantarum PBS067, Lactobacillus acidophilus PBS066, and Bifidobacterium lactis BL050; and
at least one physiologically acceptable excipient and/or carrier,
wherein:
the Lactobacillus plantarum PBS067 is present in the composition in a percentage by weight on the total weight of the probiotic combination from 0.5% to 30%;
the Lactobacillus acidophilus PBS066 is present in the composition in a percentage by weight on the total weight of the probiotic combination from 5% to 55%; and
the Bifidobacterium lactis BL050 is present in the composition in a percentage by weight on the total weight of the probiotic combination from 5% to 45%.

5. The method of claim 4, wherein the immunodepressed patient is an athlete.

6. The method of claim 4, wherein the composition is administered orally or topically.

7. A method for stimulating an immune response in a patient in need thereof, comprising administering an effective dose of a composition to the patient, wherein the composition comprises a probiotic combination comprising:
Lactobacillus plantarum PBS067, Lactobacillus acidophilus PBS066, and Bifidobacterium lactis BL050; and
at least one physiologically acceptable excipient and/or carrier,
wherein:
the Lactobacillus plantarum PBS067 is present in the composition in a percentage by weight on the total weight of the probiotic combination from 0.5% to 30%;
the Lactobacillus acidophilus PBS066 is present in the composition in a percentage by weight on the total weight of the probiotic combination from 5% to 55%; and
the Bifidobacterium lactis BL050 is present in the composition in a percentage by weight on the total weight of the probiotic combination from 5% to 45%.

8. The method of claim 7, wherein the patient is selected from the group consisting of adults, the elderly, and athletes.

9. The method of claim 7, wherein the composition is administered orally or topically.

10. The method of claim 7, wherein stimulating the immune response prevents and/or treats a viral infection.
